(19) United States
(12) Patent Application Publication
(10) Pub. No.: US 2007/0283968 A1
(43) Pub. Date: Dec. 13, 2007

(54) Method for removing calluses from feet

(76) Inventor: Jung-Bum Lee, Seoul (KR)

Correspondence Address: ROBERT E. BUSHNELL, 1522 K STREET NW, SUITE 300, WASHINGTON, DC, 20005-1202, US

(21) Appl. No.: 11/493,577

(22) Filed: Jul. 27, 2006

(30) Foreign Application Priority Data
Jun. 8, 2006 (KR) 10-2006-0051493
Jul. 6, 2006 (KR) 10-2006-0063293

(51) Int. Cl. A61B 19/00

(52) U.S. Cl. 128/898

(57) ABSTRACT

Disclosed is a method for removing calluses from feet. The method includes the steps of wrapping callus regions of the feet with a moisture-containing material, wrapping a cover on the moisture-containing material wrapping the callus regions of the feet, and removing the cover and the moisture-containing material wrapping the callus regions of the feet after a time from five minutes to one hour has lapsed. Thereby, the calluses formed on the feet can be removed in a simple and reliable way.

### CLAIM OF PRIORITY

This application makes reference to, incorporates the same herein, and claims all benefits accruing under 35 U.S.C. §119 from applications for METHOD FOR REMOVING FOOT'S HORN and METHOD FOR REMOVING CALLUSES FROM FEET earlier filed in the Korean Intellectual Property Office on 8 Jun. 2006 and 6 Jul. 2006 and there duly assigned Serial Nos. 10-2006-0051493 and 10-2006-0063293.

### BACKGROUND OF THE INVENTION

1. Field of the Invention

The present invention relates generally to a method for removing calluses from feet, and more particularly, to a method for removing calluses from feet, in which a moisture-containing material is attached to the calluses of the feet, thereby minimizing skin stimulation of the feet and simultaneously improving the chances of removing the calluses from the feet.

2. Description of the Related Art

Generally, human skin can be divided into soft parts and hard parts. Usually, the soft parts of the human skin include the face and hands that are entirely exposed to air without coming into relative contact with other objects, the legs and chest that do not come into direct contact with air by being covered with clothes while being not pressed against strongly, and so on.

In contrast, in cases of the feet on which shoes are mainly worn for walking and running and which come into long contact with stiff parts of the shoes in a standing posture, regions of the feet are hardened to form callus layers.

These callus layers gradually become thicker with the passage of time. In severe cases, the callus layers can crack, causing pain, and furthermore, germs can penetrate into the cracks, causing infections.

The callus layers have been removed in such a manner that the areas where the callus layers were formed have been soaked in hot water for a long time, and then the callus layers have been scratched off with a knife or a rough tool.

However, in the conventional method of removing the callus layers, the callus layers are generally removed in a state where the skin is not sufficiently softened. For this reason, the amount of calluses removed is small. In addition, it takes a long time to soak the callus layers in the water in order to soften the callus layers. Further, when the calluses are scratched and removed without using water, the skin may be worn out and thus damaged.

### SUMMARY OF THE INVENTION

Accordingly, the present invention has been made to solve these various problems caused in the prior art, and an object of the present invention is to provide a method for removing calluses from feet, capable of making the calluses soft and tender in a short period of time, thus improving the chances of removing the calluses from the feet.

In order to accomplish this object, there is provided a method for removing calluses from feet. The method includes the steps of wrapping callus regions of the feet with a moisture-containing material, wrapping a cover on the moisture-containing material wrapping the callus regions of the feet, and removing the cover and the moisture-containing material wrapping the callus regions of the feet after a time from five minutes to one hour has lapsed.

The method may further comprise the step of scratching off softened calluses by using a bar-like scratcher, at least one end of which is formed at an acute angle.

The method may further comprise the step of removing the remaining softened callus by using a bar-like buffer, two surfaces of which are different in roughness.

The method may further comprise the step of applying a skin cream to the regions from, which the softened calluses are removed, to thereby soften the feet.

Here, the moisture-containing material may contain sodium hydroxide within a range from 1 wt % to 5 wt %.

Further, the moisture-containing material may additionally contain bromelain within a range from 0.05 wt % to 2 wt %.

Also, the moisture-containing material may additionally contain papain within a range from 0.2 wt % to 0.4 wt %, and a-Hydroxy Acid (AHA) within a range from 4 wt % to 7 wt %.

In addition, the moisture-containing material may additionally contain glycerin within a range from 2 wt % to 3 wt %.

### BRIEF DESCRIPTION OF THE DRAWINGS

A more complete appreciation of the invention and many of the attendant advantages thereof, will be readily apparent as the same becomes better understood by reference to the following detailed description when considered in conjunction with the accompanying drawings in which like reference symbols indicate the same or similar components, wherein:

FIGS. 1A and 1B are flowcharts illustrating a process for removing calluses in a callus removing method according to the present invention;

FIG. 2A illustrates the step of wrapping a foot a moisture-containing material and a cover and removing the moisture-containing material and the cover in a callus removing method according to the present invention;

FIG. 2B illustrates the step of removing calluses by using a scratcher in a callus removing method according to the present invention;

FIG. 2C illustrates the step of removing the remaining callus by using a buffer in a callus removing method according to the present invention; and

FIG. 2D illustrates the step of softening feet by using a skin cream in a callus removing method according to the present invention.

### DETAILED DESCRIPTION OF THE INVENTION

Hereinafter, a method for removing calluses from feet according to exemplary embodiments of the present invention will be described with reference to the accompanying drawings. In the following description and drawings, the same reference numerals are used to designate the same or similar components, and so repetition of the description on the same or similar components will be omitted.

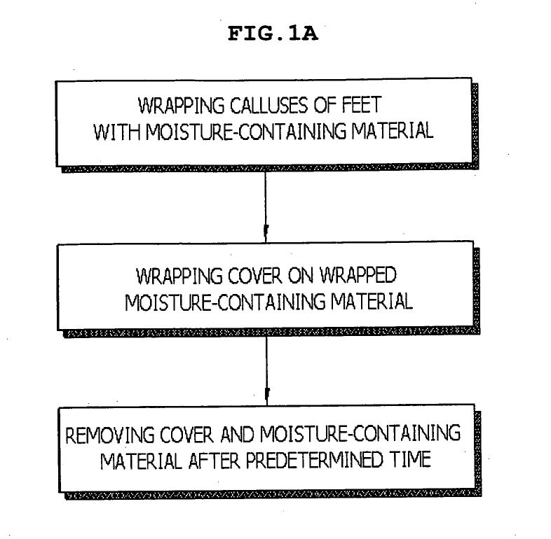

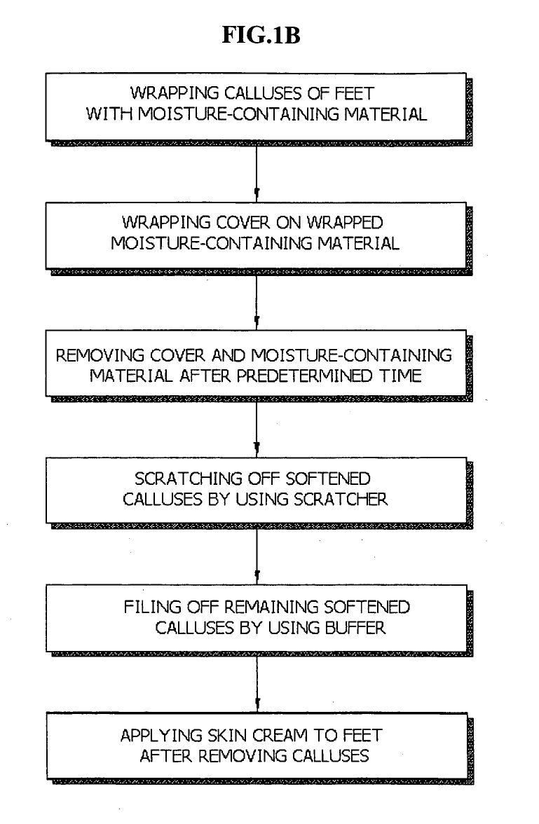

FIG. 1A is a flowchart illustrating an embodiment of a method for removing calluses from feet according to the present invention.

Referring to FIG. 1A, first, regions of the feet, on which calluses have been formed, are wrapped with a material that contains moisture and has pores thereon. The moisture-containing material may be any one of tissue, gauze, paper, non-woven fabric, fabric, and any combination thereof.

Then, a cover is wrapped on the moisture-containing material wrapping the callus regions of the feet. The cover serves to inhibit moisture and etc. from being evaporated from the moisture-containing material and maintain a sealed state.

Then, after a predetermined time, for instance, from five minutes to one hour, the moisture-containing material and cover wrapping the callus regions of the feet are removed. At this time, a time for which the moisture-containing material and cover are wrapped on the callus regions can be selected within a range from five minutes to one hour, and preferably from ten minutes to thirty minutes. For the purpose of efficient callus removal, it is most preferable to remove the moisture-containing material and cover after 15 minutes. After a predetermined time, the moisture-containing material and cover are removed, and the calluses of the feet are softened. In this state, the calluses can be removed with one's own hands,

FIG. 1B is a flowchart illustrating another embodiment of a method for removing calluses from feet according to the present invention.

The calluses may be removed as in FIG. 1A. However, in order to more efficiently remove the softened calluses, a method for removing the softened calluses using a buffer and a scratcher will be described.

The scratcher is used to more efficiently remove the softened calluses after the cover and the moisture-containing material are removed. The softened calluses are scratched off by the scratcher, one end of which is formed at an acute angle. Herein, the scratcher refers to a bar-like thin stick which is made of a plastic material, and is constructed to easily remove the calluses because one region is inclined at a predetermined angle, for example, 45°.

Further, in order to more efficiently remove the softened calluses, a buffer can be used. The remaining calluses, after scratching, are filed off by the buffer, which has a bar shape and two surfaces of different roughness. Herein, the buffer means a flat bar whose front and rear surfaces are different in roughness and which has a constant thickness.

Then, a skin cream is applied to the regions where the softened calluses have been scratched and filed off, and then the feet are massaged softly. Because the skin cream contains moisture, it can supply the feet, which are rough after scratching off the calluses, with moisture, and thereby soften the feet.

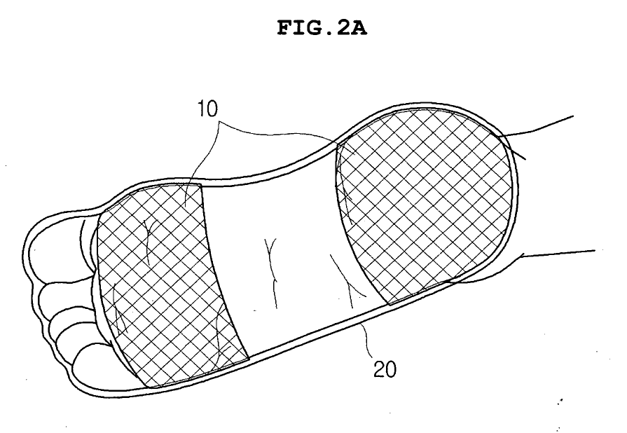

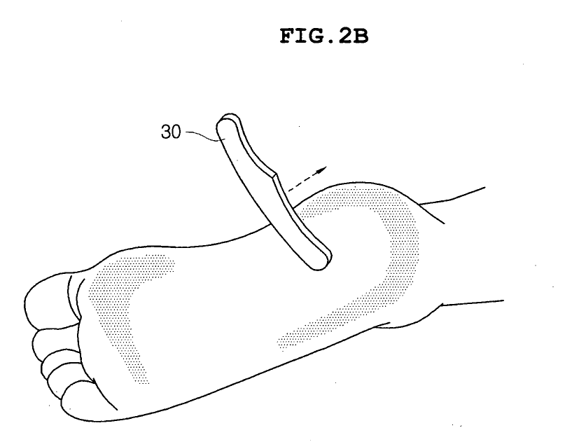

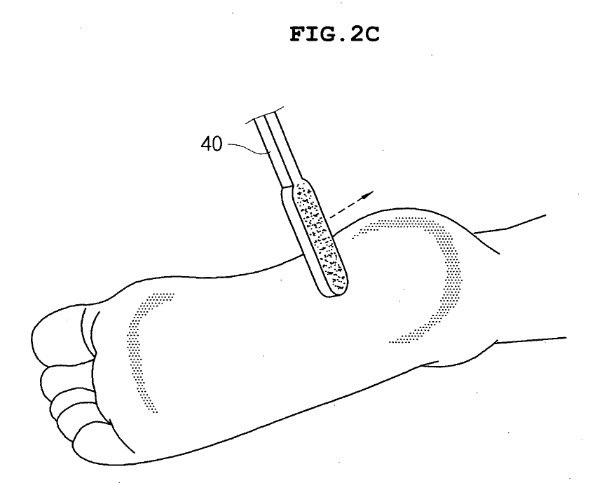

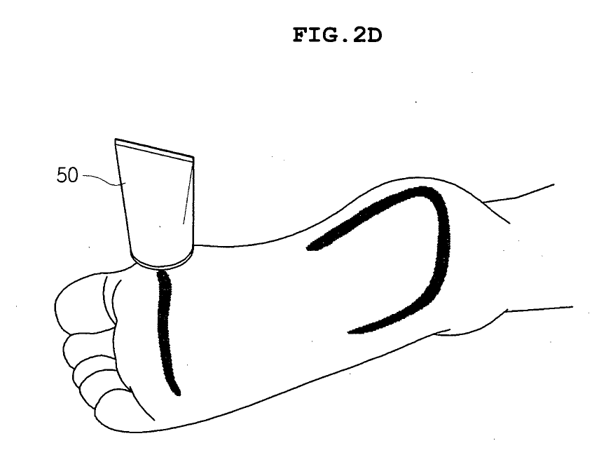

Hereinafter, a method for removing the calluses will be described step by step. First, an embodiment of removing the calluses using a moisture-containing material and a cover will be described with reference to FIG. 2A.

As illustrated in FIG. 2A, first, regions of the feet, on which the calluses have formed, are wrapped with the moisture-containing material 10 on which pores are formed. Here, the moisture-containing material may be any one of tissue, gauze, paper, non-woven fabric and fabric. It should be understood that the moisture-containing material can be selected from a material other than the illustrated materials.

Then, the cover 20 is wrapped on the moisture-containing material 10 which has been wrapped on the callus regions of the feet. Thereby, the cover 20 inhibits moisture and other components from being evaporated from the moisture-containing material 10, and helps the components of the moisture-containing material 10 to permeate well into the callus regions of the feet. At this time, anything other than the cover 20 will do, if it can prevent the moisture of the moisture-containing material 10 from being evaporated.

After a predetermined time, for instance, from five minutes to one hour, has lapsed with the moisture-containing material 10 and the cover 20 wrapped, the cover 20 and the moisture-containing material 10, which wrap the callus regions of the feet, are removed. A time for which the callus regions are wrapped with the moisture-containing material 10 and the cover 20 is preferably within a range from ten minutes to thirty minutes, and most preferably about fifteen minutes. When the cover 20 and the moisture-containing material 10 are removed, the calluses of feet are in a softened state. In this state, the calluses can be easily removed with one's own hands.

Next, an embodiment of removing the calluses by using a scratcher and a buffer will be described with reference to FIGS. 2B through 2D.

FIG. 2B illustrates the step of removing calluses by using a scratcher in a callus removing method according to the present invention.

In order to more easily remove the calluses softened after the cover 20 and the moisture-containing material 10 are removed, the calluses are scratched off by the scratcher 30 on which one end is formed at an acute angle, as illustrated in FIG. 2B. A part of the scratcher, in particular, the part where the acute angle is formed, is brought into close contact with the softened callus region of the foot, and then the callus is scratched off. Herein, the scratcher 30 is a tool for easily scratching off the callus, and is made of a plastic material. However, the scratcher may be made of another material.

FIG. 2C illustrates the step of removing the remaining calluses by using a buffer in a callus removing method according to the present invention.

As illustrated in FIG. 2C, the remaining calluses of the feet after the softened calluses are removed by the scracher 30 can be removed by the buffer 40. The buffer 40 has two surfaces of different roughness, so that it can remove the remaining fine calluses. The remaining calluses of the feet are first filed off by one surface of the buffer 40 which has a larger roughness and then by the other surface of the buffer 40 which has a finer roughness, so that the remaining calluses of the feet can be completely removed.

FIG. 2D illustrates the step of softly massaging feet by using a skin cream in a callus removing method according to the present invention.

As illustrated in FIG. 2D, after the calluses of feet are fully removed by the scratcher 30 and the buffer 40, the regions from which the calluses of feet are removed are not in a soft state. In order to soften the feet from which the calluses are removed, the skin cream, in which moisture is contained, is uniformly applied to the regions from which the calluses have been removed.

Through these processes, the calluses of feet can be removed easily and simply.

Meanwhile, the moisture-containing material 10 used for removing the calluses can contain sodium hydroxide within a range from 1 wt % to 5 wt %.

At this time, the sodium hydroxide softens the skin by adjusting pH. When the content of sodium hydroxide is less than 1 wt %, a function of softening the skin is lowered. In contrast, when the content of sodium hydroxide is more than 5 wt %, this is meaningless because there is no particular improvement in terms of effects.

Further, the moisture-containing material 10 used for removing the callus may additionally contain bromelain within a range from 0.05 wt % to 2 wt %. The bromelain also serves to soften the skin. When the content of bromelain is less than 0.05 wt %, the effect of softening the skin shows slightly. In contrast, when the content of bromelain is more than 2 wt %, this is unfavorable because mixablity with other components is deteriorated.

Further, the moisture-containing material 10 used for removing the calluses may additionally contain papain within a range from 0.2 wt % to 0.4 wt %. The reason of adding papain is the papain helps serving to soften the skin. The content of papain preferably has the range from 0.2 wt % to 0.4 wt %. When the content of papain is less than 0.2 wt %, the effect of softening the skin does not show properly. In contrast, when the content of papain is more than 0.4 wt %, other effects of the skin softener for the calluses used in the present invention may be deteriorated.

Further, the moisture-containing material 10 used for removing the calluses may additionally contain α-Hydroxy Acid (AHA) within a range from 4 wt % to 7 wt %, and glycerin within a range from 2 wt % to 3 wt %.

The AHA is a compound where an alcohol or hydroxyl group is added to carbon located at an alpha (α) position of carboxylic acid. The AHA lowers ion binding energy of callus cells to remove an excessive callus or facilitate formation of a new skin cell, and thereby shortens a cycle of a keratinization process prolonged with aging. As a result, the AHA is to facilitate activity of the skin cells in order to improve the skin. Also, the AHA facilitates creation of collagens and elastics by fibroblasts, and the resulting creation of mucopolysacharide that makes the skin elastic and soft is an important extra cellular matrix component of the dermis, thereby increasing the moisture level of the skin.

When the content of AHA is less than 4 wt %, the above-mentioned effects caused by the AHA are not properly exerted. However, when the content of AHA is more than 7 wt %, there is a possibility of generating soreness, burn, and so on because it is irritative to the skin.

As examples of the AHA, there are latic acid, pyroglutamic acid, citric acid, glycolic acid, malic acid, pyruvic acid, tartaric acid, and so on. The AHA used as a composition of the present invention can make use of the glycolic acid obtained from sugar canes or sugar beets, the latic acid contained in sour milk, the malic acid found in unripe apples, the tartaric acid obtained form sour grapes, the citric acid contained in citrus fruits, and mixtures thereof.

Meanwhile, the glycerin is helpful to a moisture-bearing effect of the skin. When the content of glycerin added is less than 2 wt %, it is difficult to properly exert the moisturization effect on the skin caused by the addition of the glycerin. Further, when the content of glycerin added is more than 3 wt %, the other functions of the present invention can be deteriorated.

As can be seen from the foregoing, the callus removing method is described in detail focusing on the configuration of the present invention.

With the above-described configuration, the calluses can be removed when the skin is sufficiently softened, so that the calluses of the feet can be easily removed. Further, it is possible to decrease the damage of the skin when the calluses are removed.

Although exemplary embodiments of the present invention has been described for illustrative purposes, those skilled in the art will appreciate that various modifications, additions and substitutions are possible, without departing from the scope and spirit of the invention as disclosed in the accompanying claims.

What is claimed is:

1. A method for removing calluses from feet, the method comprising the steps of:
wrapping callus regions of the feet with a moisture-containing material;
wrapping a cover on the moisture-containing material wrapping the callus regions of the feet; and
removing the cover and the moisture-containing material wrapping the callus regions of the feet after a time from five minutes to one hour has lapsed.

2. The method as claimed in claim 1, further comprising the step of scratching off softened calluses by using a bar-like scratcher, at least one end of which is formed at an acute angle.

3. The method as claimed in claim 2, further comprising the step of removing the remaining softened callus by using a bar-like buffer, two surfaces of which are different in roughness.

4. The method as claimed in claim 3, further comprising the step of applying a skin cream to the regions from, which the softened calluses are removed, to thereby soften the feet.

5. The method as claimed in claim 1, wherein the moisture-containing material includes any one of tissue, gauze, paper, non-woven fabric and fabric.

6. The method as claimed in claim 2, wherein the moisture-containing material includes any one of tissue, gauze, paper, non-woven fabric and fabric.

7. The method as claimed in claim 3, wherein the moisture-containing material includes any one of tissue, gauze, paper, non-woven fabric and fabric.

8. The method as claimed in claim 4, wherein the moisture-containing material includes any one of tissue, gauze, paper, non-woven fabric and fabric.

9. The method as claimed in claim 5, wherein the moisture-containing material contains sodium hydroxide within a range from 1 wt % to 5 wt %.

10. The method as claimed in claim 9, wherein the moisture-containing material additionally contains bromelain within a range from 0.05 wt % to 2 wt %.

11. The method as claimed in claim 10, wherein the moisture-containing material additionally contains papain within a range from 0.2 wt % to 0.4 wt %.

12. The method as claimed in claim 11, wherein the moisture-containing material additionally contains α-Hydroxy Acid (AHA) within a range from 4 wt % to 7 wt %.

13. The method as claimed in claim 12, wherein the moisture-containing material additionally contains glycerin within a range from 2 wt % to 3 wt %.
